(19) United States
(12) Patent Application Publication
(10) Pub. No.: US 2006/0135499 A1
(43) Pub. Date: Jun. 22, 2006

(54) Method of achieving accelerated weight loss by administration of a weight loss accelerating agent to a dieting mammal

(76) Inventor: John L Zenk, Minnetrista, MN (US)

Correspondence Address: SHERRILL LAW OFFICES, 4756 BANNING AVE, SUITE 212, WHITE BEAR LAKE, MN, 55110-3205, US

(21) Appl. No.: 10/541,781

(22) PCT Filed: Jan. 12, 2004
(86) PCT No.: PCT/US04/00537

§ 371 Date: Feb. 6, 2006

Related U.S. Application Data
(60) Provisional application No. 60/439,753, filed on Jan. 13, 2003.

(51) Int. Cl. A61K 31/57

(52) U.S. Cl. 514/178

(57) ABSTRACT

Accelerating weight loss by admtinistering to a dieting mammal the weight loss accelerating agent 7-oxo DHEA or a pro-drug thereof incapable of in vivo conversion to testosterone.

This application claims the benefit of U.S. Provisional Application No. 60/439,753, filed Jan. 13, 2003.

### FIELD OF INVENTION

The invention relates to methods of achieving weight loss.

### BACKGROUND

The steroid Δ5-androstene-3-ol-7,17-dione (7-oxo DHEA) is believed to stimulate various beneficial biological responses including (i) inducing the synthesis of various thermogenic enzymes which are effective for regulating metabolism and thereby promoting weight control without affecting caloric intake, and (ii) inducing the synthesis of the major thyroid hormone triiodothyronine (T₃) which is effective for increasing the basal metabolic rate and thereby promoting weight control without affecting caloric intake.

The ability of 7-oxo DHEA to promote weight control is widely believed to be mediated through enhanced thermogenesis (conversion of foodstuffs to heat energy rather than chemical energy such as ATP and/or triacylglycerides). The thermogenic effect mediated by 7-oxo DHEA is believed to result from the ability of 7-oxo DHEA to stimulate the synthesis of thermogenic enzymes including mitochondrial glycerol 3-phosphate dehydrogenase (G3P-DH), cytosolic malic enzyme (ME) and fatty acyl CoA oxidase. Such enzymes tend to reduce the efficiency of energy metabolism within the body.

While highly effective for safely promoting weight control, a continuing need exists to accelerate the weight loss effect achieved with 7-oxo DHEA.

### SUMMARY OF THE INVENTION

Weight loss can be accelerated during dieting by the administration of a weight loss accelerating agent while dieting. The weight loss accelerating agent is 7-oxo DHEA or a pro-drug thereof incapable of in vivo conversion to testosterone.

### DETAILED DESCRIPTION OF A PREFERRED EMBODIMENT

Definitions

As utilized herein, including the claims, the term “dieting” means eating and drinking sparingly with the intent to lose weight.

As utilized herein, including the claims, the term “7-oxo DHEA” means Δ5-androstene-3-ol-7,17-dione.

As utilized herein, including the claims, the term “3-acetyl 7-oxo DHEA” means Δ5-androstene-3-acetoxy-7,17-dione.

Description

I have surprisingly discovered that 7-oxo DHEA is effective for accelerating the weight loss achievable by dieting. Without intending to be limited to any particular theory, I believe that the administration of 7-oxo DHEA to a dieting mammal is effective for accelerating weight loss because 7-oxo DHEA modulates the metabolism of the dieting mammal. It is widely believed that dieting is an ineffective means for achieving weight loss because the body reacts to the reduced caloric intake by slowing down the metabolism of the dieter. By modulating the metabolism of the dieting mammal, 7-oxo DHEA would be effective for preventing or at least moderating any diet-induced decrease in the metabolism and thereby accelerate weight loss achievable by dieting.

The Weight Loss Accelerating Agent

The weight loss accelerating agent effective for accelerating weight loss when combined with dieting is the steroid Δ5-androstene-3β-ol-7,17 dione (7-oxo DHEA). 7-oxo DHEA is a derivative of dehydroepiandrosterone (DHEA). 7-oxo DHEA does not appreciably stimulate, increase or otherwise enhance the production of sex hormones. The steroid is commercially available from a number of sources including Steraloids, Inc. of Wilton, N.H. A number of procedures are available for synthesizing Δ5-androstene-3β-ol-7,17 dione from DHEA, with one such procedure described in U.S. Pat. No. 5,296,481.

Pro-drugs of 7-oxo DHEA (i.e., compounds readily metabolized in vivo to the active 7-oxo DHEA) may also be usefully employed. One example of a pro-drug is the commercially available Δ5-androstene-3β-acetyl-7,17 dione (3-acetyl 7-oxo DHEA). The 3β-acetyl group is hydrolyzed in vivo by esterases located in the blood and various tissue to produce the active 7-oxo DHEA, and is believed to be less susceptible to oxidation during the manufacturing process relative to 7-oxo DHEA. Other suitable pro-drugs include Δ5-androstene-3β,17β-diol-7-one, Δ5-androstene-3β,7α-diol-17-one, Δ5-androstene-3β,7β-diol-17-one and the corresponding acetyl esters of these steroids.

Administration

Administration Route

The weight loss accelerating agent can be administered by virtually any of the commonly accepted practices for the administration of pharmaceutical preparations including specifically, but not exclusively, mucosal administration, oral consumption, ocular administration, subcutaneous injection, transdermal administration, etc. Oral administration is generally preferred.

Mucosal administration of the weight loss accelerating agent includes such routes as buccal, endotracheal, nasal, pharyngeal, rectal, sublingual, vaginal, etc. For administration through the buccal/sublingual/pharyngeal/endotracheal mucosal, the weight loss accelerating agent may be formulated as an emulsion, gum, lozenge, spray, tablet or an inclusion complex such as cyclodextrin inclusion complexes. Nasal administration is conveniently conducted through the use of a sniffing powder or nasal spray. For rectal and vaginal administration the weight loss accelerating agent may be formulated as a cream, douche, enema or suppository.

Oral consumption of the weight loss accelerating agent may be effected by incorporating the weight loss accelerating agent into a food or drink, or formulating the weight loss accelerating agent into a chewable or swallowable tablet or capsule.

Ocular administration may be effected by incorporating the weight loss accelerating agent into a solution or suspension adapted for ocular application such as drops or sprays.

Subcutaneous administration involves incorporating the weight loss accelerating agent into a pharmaceutically acceptable and injectable carrier.

For transdermal administration, the weight loss accelerating agent may be conveniently incorporated into a lipophilic carrier and formulated as a topical crème or adhesive patch.

Dose Rate

The range of dosages and dose rates effective for achieving the desired accelerative weight loss effect may be determined in accordance with standard industry practices.

I/we claim:

1. A method of achieving accelerated weight loss comprising administration of a weight loss accelerating agent to a dieting mammal wherein the weight loss accelerating agent is 7-oxo DHEA or a pro-drug thereof incapable of in vivo conversion to testosterone.

2. The method of claim 1 wherein the weight loss accelerating agent is administered orally.

3. The method of claim 2 wherein the weight loss accelerating agent is administered at least once daily.

4. The method of claim 1 wherein the dieting mammal is a human.

5. The method of claim 2 wherein the dieting mammal is a human.

6. The method of claim 3 wherein the dieting mammal is a human.

7. The method of claim 4 wherein the weight loss accelerating agent is 3-acetyl 7-oxo DHEA or 3-ester thereof.

8. The method of claim 5 wherein the weight loss accelerating agent is 3-acetyl 7-oxo DHEA or 3-ester thereof.

9. The method of claim 6 wherein the weight loss accelerating agent is 3-acetyl 7-oxo DHEA or 3-ester thereof.
